(19) United States
(12) Patent Application Publication
(10) Pub. No.: US 2014/0114065 A1
(43) Pub. Date: Apr. 24, 2014

(54) ANHYDROUS LINEZOLID CRYSTALLINE FORM-II

(71) Applicants: Raghu Mitra Alla, Hyderabad (IN); Ajay Kumar Dubey, Hyderabad (IN); Aruna Kumari Sirigiri, Hyderabad (IN); Naga Karna Kurmar Mareedu, Krishna (IN)

(72) Inventors: Raghu Mitra Alla, Hyderabad (IN); Ajay Kumar Dubey, Hyderabad (IN); Aruna Kumari Sirigiri, Hyderabad (IN); Naga Karna Kurmar Mareedu, Krishna (IN)

(73) Assignee: Lee Phama Limited, Hyderabad (IN)

(21) Appl. No.: 13/820,565

(22) PCT Filed: Feb. 21, 2012
(86) PCT No.: PCT/IN2012/000120

§ 371 Date: Mar. 4, 2013

(30) Foreign Application Priority Data
Feb. 24, 2011 (IN) 2450/CHE/2010

(51) Int. Cl. C07D 263/24

(52) U.S. Cl. CPC C07D 263/24
USPC 544/137

(57) ABSTRACT

The present invention relates to oxazolidinone antibacterial agent anhydrous Linezolid crystalline Form-II.

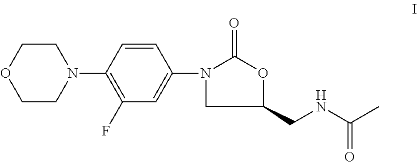 [SMILES: CC(=O)NC[C@H]1CN(c2ccc(N3CCOCC3)c(F)c2)C(=O)O1]

The anhydrous Linezolid crystalline Form-II of the invention comprises less than 0.5% of water content. The anhydrous Linezolid crystalline Form-II of the invention is characterized by the XRPD spectrum and IR spectrum described in the specification.

### FIELD OF THE INVENTION

Present invention relates to novel anhydrous Linezolid crystalline Form-II. More particularly, the invention relates to anhydrous Linezolid crystalline Form-II consisting of less than 0.5% w/w of water content.

### BACKGROUND OF THE INVENTION

U.S. Pat. No. 5,688,792 discloses the antibacterial agent Linezolid as well as a process for its preparation. There are many other references for the preparation and isolation of Linezolid. J. Med. Chem 39(3), 673-679 (1996) reports that Linezolid was re-crystallized from ethyl acetate &. hexane as white crystals, with melting point of 181.5-182.5° C. It also sets for the IR spectrum as 3284, 3092, 1753, 1728, 1649, 1565, 1519, 1447 & 1435.

Tetrahedron Lett. 40 (26) 4855 (1999) and U.S. Pat. No. 5,837,870, WO99/24393 disclose Linezolid and process to prepare the Linezolid. Both the publications do not set forth the melting point or IR spectrum.

U.S. Pat. No. 6,559,305 B1 discloses the preparation of Linezolid crystal form-II by mixing greater than 98% enantiomeric pure (S)-3-(3-fluoro-4-morpholinophenyl)-2-oxo-5-oxazolidinyl methyl acetamide in ethyl acetate solvent at temperature below of about 80° C. and separating the Linezolid crystal form-II.

US20090062534 A1 discloses the novel Linezolid crystalline hemihydrated form with water content ranging from 2.5-3.5% w/w and to addition salts of Linezolid.

### OBJECT OF THE INVENTION The primary object of the invention is to provide a novel anhydrous crystalline Form of Linezolid.

Another object of the invention is to provide a novel anhydrous Linezolid crystalline Form-II.

Another object of the invention is to provide a method for preparation of the anhydrous Linezolid crystalline Form-II.

### SUMMARY OF THE INVENTION

Accordingly, to meet the above objectives, present invention provides novel process to prepare anhydrous Linezolid crystal form-II, which comprises:
(i) Dissolving or mixing (S)-3-(3fluoro-4-morpholinophenyl)-2oxo-5-oxazolidinyl methyl acetamide having enantiomeric impurity <0.5% in a solvent at a preferable temperature of about 90-95° C.;
(ii) Precipitating anhydrous Linezolid crystal form-II having enantiomeric impurity <0.5% and purity by HPLC as per ICH limits from the solvent.

The solvent used in the above processes is selected from the group consisting of esters such as n-butyl acetate, alcohols such as sec. butanol and tert. butanol to yield directly Linezolid crystalline form-II anhydrous.

### BRIEF DESCRIPTION OF THE DRAWINGS

FIG. 1: Shows the X-ray diffraction spectrum of anhydrous Linezolid crystalline form-II (Example-1)

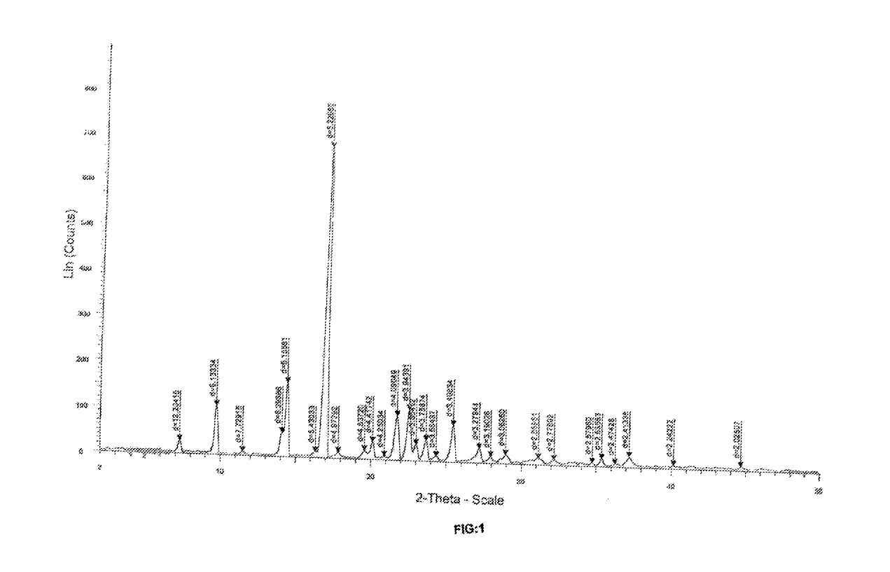

FIG. 2: Shows the IR spectrum of anhydrous Linezolid crystalline form-II (Example 1)

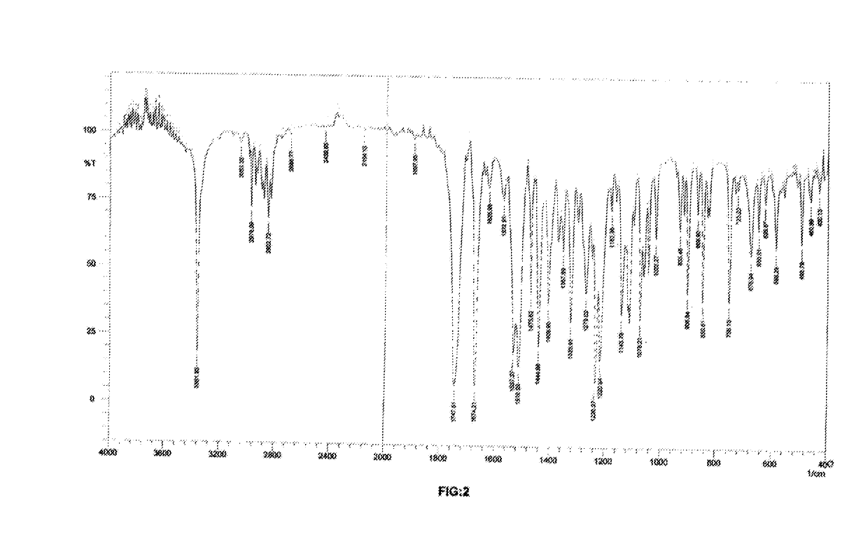

FIG. 3: Shows the X-ray diffraction spectrum of anhydrous Linezolid crystalline form-II (Example 2)

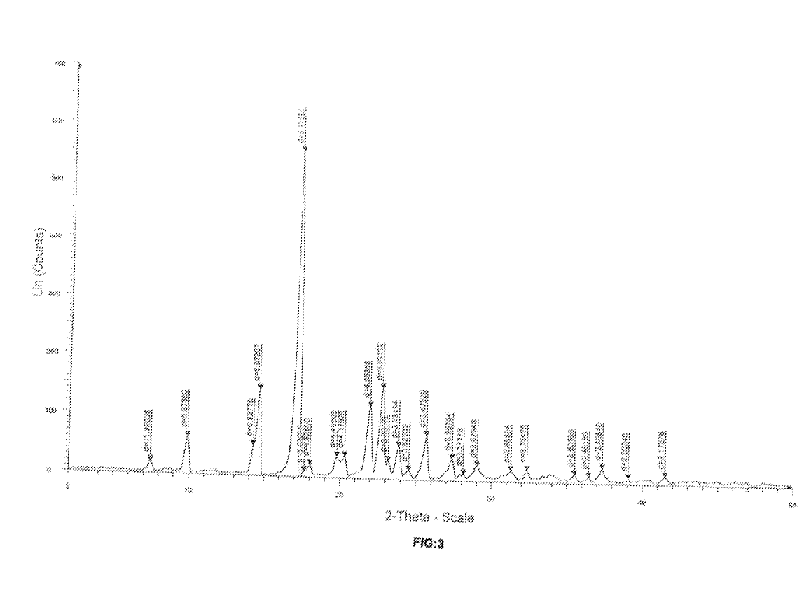

FIG. 4: Shows the IR spectrum of anhydrous Linezolid crystalline form-II (Example 2)

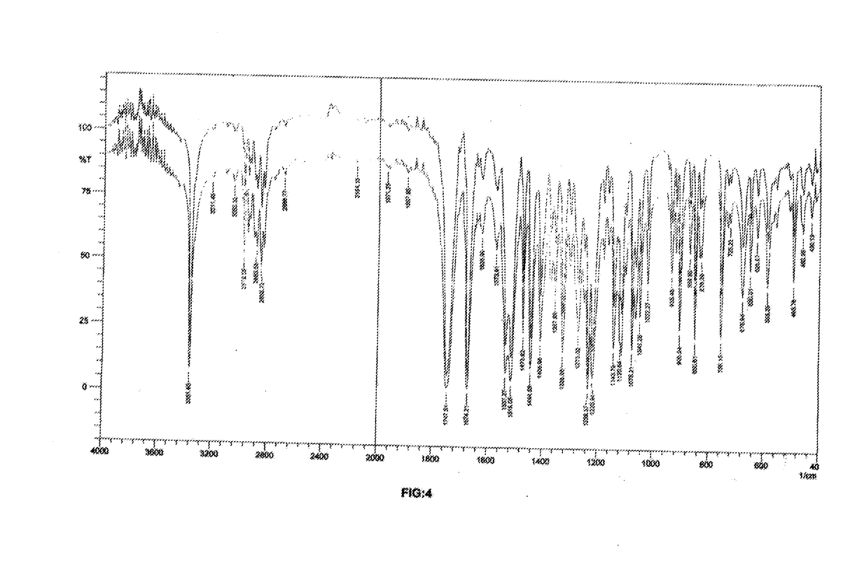

FIG. 5: Shows the X-ray diffraction spectrum of anhydrous Linezolid crystalline form-II (Example 3)

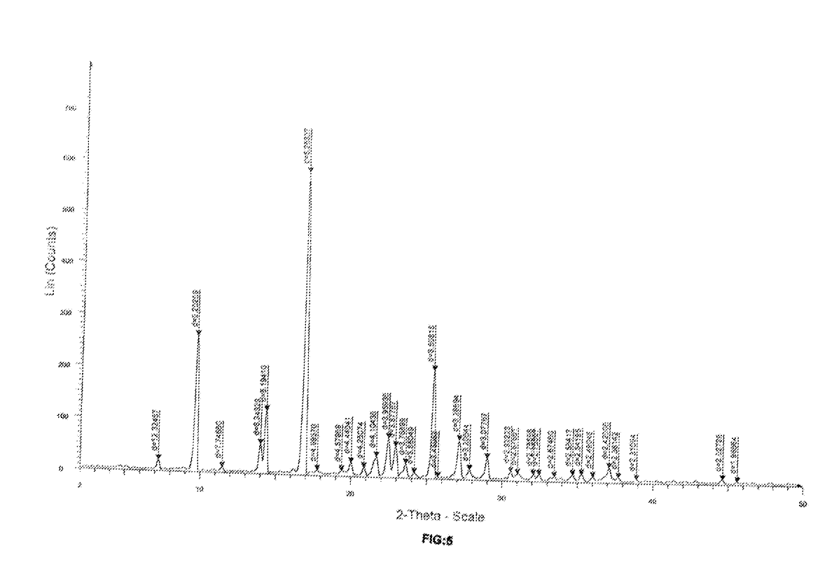

FIG. 6: Shows the IR spectrum of anhydrous Linezolid crystalline form-II (Example 3)

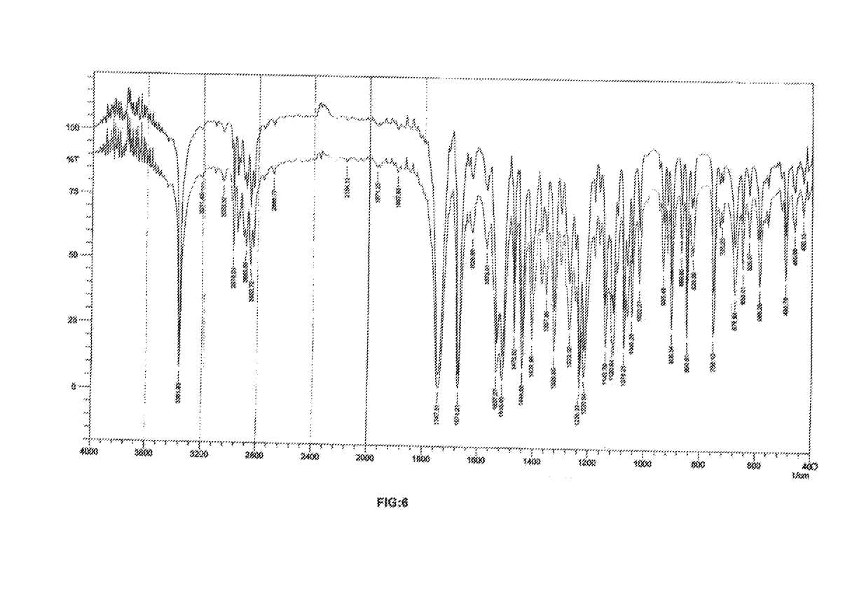

### DETAILED DESCRIPTION OF THE INVENTION

While the invention will now he described in detail in connection with certain preferred and optional embodiments. So that various aspects there of may be more fully understood and appreciated. It is not intended to limit the invention to these particular embodiments. Detailed description of the embodiment, which is outlined in a broad sense and featured in the invention, so that those skilled in the art may better understand the detailed process.

According to the present invention, novel anhydrous Linezolid crystalline form-II can be prepared by dissolving or mixing of Linezolid in suitable solvent followed by heating and precipitating from solvent upon cooling.

The solvent used in the above process is selected from the group consisting of esters and alcohols such as n-butyl acetate, sec. butanol and tert. butanol.

Thus, the processes of the present invention are reliable, convenient and easily reproducible on industrial scale and give substantially identical anhydrous crystalline form-II of Linezolid, which is exemplified through the following examples.

### EXAMPLES

### Preparation of Anhydrous Linezolid Crystalline Form-II

### Example-1

Linezolid having an enantiomeric impurity less than 0.5% (10 gr) is suspended with n-butyl acetate (200 ml). Heated the suspension to 85-90° C. and the mixture is stirred for 40-45 min. Slowly cool the mixture to 25-30° C. and stirred for 30 min and then the mixture is cooled to 0-5° C., and stirred for 60 min. The precipitated solids are filtered to give anhydrous Linezolid crystal form-II. The resulting crystalline form-II, complies the water content <0.5% and it is characterized by XRPD spectrum and IR spectrum.

### Example-2

Linezolid having enantiomeric impurity less than 0.5% (10 gr) is suspended with sec. butanol (100 ml). Heated the suspension to 85-90° C. and the clear solution is stirred for 40-45 min. Slowly cool the solution to 25-30° C. and stirred for 30 min and then the mixture is cooled to 0-5° C. and stirred for 60 min. The precipitated solids are filtered to give anhydrous Linezolid crystalline form-II. The resulting crystalline form-II complies the water content <0.5% and it is characterized by XRPD spectrum and IR spectrum.

### Example-3

Linezolid having an enantiomeric impurity less than 0.5% (10 gr) is suspended with tertiary butanol (200 ml). Heated the suspension to 85-90° C. and the mixture is stirred for 40-45 min. Slowly cool the mixture to 25-30° C. and stirred for 60 min. The precipitated solids are filtered to give anhydrous Linezolid crystalline form-II. The resulting crystalline form-II complies the water content <0.5% and it is characterized by XRPD spectrum and IR spectrum.

We claim:

1. Anhydrous Linezolid crystalline Form-II comprising <0.5% w/w of water content.

2. Anhydrous Linezolid crystalline Form-II as claimed in claim 1, wherein the anhydrous Linezolid crystalline Form-II has XRPD spectrum of 7.10, 9.54, 13.88, 14.23, 16.18, 16.79, 17.69, 19.41, 19.69, 19.93, 21.61, 22.39, 22.84, 23.52, 24.16, 25.28, 26.66, 27.01 and 27.77±0.2° in 2Θ.

3. Anhydrous Linezolid crystalline Form-II as claimed in claim 1, wherein the anhydrous Linezolid crystalline form-II has IR spectrum 3364, 1748, 1675, 1537, 1517, 1445, 1410, 1401, 1358, 1329, 1287, 1274, 1253, 1237, 1221, 1145, 1130, 1123, 1116, 1078, 1066, 1049, 907, 852 and 758 cm″1.

4. Anhydrous Linezolid crystalline Form-II as claimed in claim 1, comprising synthesizing (S)-3-(3-fluoro-4-morpholinophenyl)-2-oxo-5-oxazolidinyl methyl acetamide having enantiomeric purity <0.5%.

5. Anhydrous Linezolid crystalline Form-II as claimed in claim 1, comprising synthesizing (S)-3-(3-fluoro-4-morpholinophenyl)-2-oxo-5-oxazolidinyl methyl acetamide having impurity by HPLC <0.1%.

6. A process for preparation of anhydrous Linezolid crystalline Form-II as claimed in claim 1, comprising
mixing the (S)-3-(3-fluoro-4-morpholinophenyl)-2-oxo-5-oxazolidinyl methyl acetamide in a solvent at a temperature about 90-95° C.

7. A process according to claim 6, wherein the solvent is selected from the group consisting of secondary butanol, tertiary butanol and n-butyl acetate.

8. A process according to claim 6, wherein the solvent is n-butyl acetate.

9. A process according to claim 6, wherein the (S)-3-(3-fluoro-4-morpholinophenyl)-2-oxo-5-oxazolidinyl methyl acetamide is mixed for at least 40-45 minutes.
